(19) United States
(12) Patent Application Publication
(10) Pub. No.: US 2010/0136147 A1
(43) Pub. Date: Jun. 3, 2010

(54) DIET SUPPLEMENT FOR CAUSING WEIGHT LOSS

(75) Inventors: Marvin A. Heuer, Oakville (CA); Ken Clement, Oakville (CA); Shan Chaudhuri, Oakville (CA)

Correspondence Address: FITZPATRICK CELLA HARPER & SCINTO, 1290 Avenue of the Americas, NEW YORK, NY, 10104-3800, US

(73) Assignee: Northern Innovations and Formulations Corp., Oakville, ON (CA)

(21) Appl. No.: 12/440,082

(22) PCT Filed: Sep. 8, 2006
(86) PCT No.: PCT/CA06/01473

§ 371 Date: Jun. 9, 2009

(51) Int. Cl. A61K 36/82

(52) U.S. Cl. 424/729

(57) ABSTRACT

A dietary supplement and method for supporting weight loss and reducing the waist circumference of an individual, the dietary supplement comprising an extract of Cissus Quadrangularis stem and leaf, an extract of Green Tea leaf and an effective amount of Caffeine Anhydrous. The dietary supplement may further comprise at least one of Chromium Polynicotinate, an extract of Soy Bean Albumin, Pyridoxine Hydrochloride, L-selenomethionine, Cyanocobalmin, Yohimbine Hydrochloride and Folic Acid.

### FIELD OF THE INVENTION

The present invention relates to a diet supplement for supporting weight loss. More specifically, the present invention relates to a diet supplement containing an effective amount of anhydrous caffeine that is relative to an amount of caffeine contained in extracts of Green Tea Leaves.

### BACKGROUND OF THE INVENTION

Glucose clearance is a measure of the efficiency of glucose transport. More specifically, it is related to glucose uptake or utilization and plasma glucose concentration. The uptake of glucose by cells is mediated through a family of glucose transporters (GLUTs). Individuals with type-2 diabetes have delayed glucose clearance compared to normal individuals with similar levels of insulin. This delayed glucose clearance has been linked to the development of diabetes, glucose toxicity as well as insulin resistance (Woerle H J, Szoke E, Meyer C, Dostou J M, Wittlin S D, Gosmanov N R, Welle S L, Gerich J E. Mechanisms for abnormal postprandial glucose metabolism in type 2 diabetes. Am J Physiol Endocrinol Metab, 2006. 290(1): p. E67-E77). Delayed glucose clearance is also associated with increased adiposity and elevated serum cholesterol and triglyceride levels (Krishnan R K, Evans W J, Kirwan J P. Glucose clearance is delayed after hyperglycemia in healthy elderly men. J Nutr, 2003. 133(7): p. 2363-6). Furthermore, weight gain has been shown to impair glucose clearance in children (Crowther N J, Cameron N, Trusler J, Gray I P. Association between poor glucose tolerance and rapid post natal weight gain in seven-year-old children. Diabetologia, 1998. 41(10): p. 1163-7).

There are several different blood lipid parameters used to assess a person's health and make predictions regarding an individual's physical well-being. Low density lipoproteins (LDL) are considered a ‘bad’ type of cholesterol, whereas high density lipoproteins (HDL) are ‘good’ cholesterol. Levels of serum HDL's have long been associated with good health (Nikkilä EA: Letter: Serum high-density-lipoprotein and coronary heart-disease. Lancet, 1976. 2:320). While cholesterol is essential for cell membranes in addition to being a precursor for bile acid and steroid hormone synthesis, it is poorly soluble in blood and requires the assistance of transport molecules. Lipoproteins provide this function to act as vehicles for the transport of cholesterol. In addition to the specific proteins with which HDL's and LDL's are comprised, they also differ in size and density. An HDL is the smallest lipoprotein and is largely involved in the removal of excess cholesterol, where it may disposed of in the liver (Kwiterovich P O Jr. The metabolic pathways of high-density lipoprotein, low-density lipoprotein, and triglycerides: a current review. Am J. Cardiol. 2000, 86(12A):5L-10L). LDL's, on the other hand are larger than HDL's and are the main transporter of cholesterol within the blood (Cromwell W C, Otvos J D. Low-density lipoprotein particle number and risk for cardiovascular disease. Curr Atheroscler Rep. 2004, 6(5):381-7). Blood transports cholesterol to cells for use, including the arteries, where high levels of cholesterol may lead to the formation of plaques resulting in cardiovascular disease. One of the most accurate and accepted predictors of health measures is the HDL/LDL ratio (Lemieux I, Lamarche B, Couillard C, Pascot A, Cantin B, Bergeron J, Dagenais G R, Despres J P. Total cholesterol/HDL cholesterol ratio vs LDL cholesterol/HDL cholesterol ratio as indices of ischemic heart disease risk in men: the Quebec Cardiovascular Study. Arch Intern Med, 2001. 161(22): p. 2685-92). Body weight reduction, through dieting, has been shown to favorably change this ratio (Dattilo A M, Kris-Etherton P M. Effects of weight reduction on blood lipids and lipoproteins: a meta-analysis. Am J Clin Nutr, 1992. 56(2): p. 320-8). Moreover, serum triglyceride levels also have an accepted prognostic value for heart disease (Manninen V, Tenkanen L, Koskinen P, Huttunen J K, Manttari M, Heinonen O P, Frick M H. Joint effects of serum triglyceride and LDL cholesterol and HDL cholesterol concentrations on coronary heart disease risk in the Helsinki Heart Study. Implications for treatment. Circulation, 1992. 85(1): p. 37-45). Triglycerides are a common form of fat in the body. Triglycerides can be directly obtained from ingested food or formed in the body as a method of energy storage when energy supply exceeds energy requirements. Studies have shown a relationship between reduction in body weight and decreases in serum triglyceride levels (Nordmann A J, Nordmann A, Briel M, Keller U, Yancy W S Jr, Brehm B J, Bucher H C. Effects of low-carbohydrate vs low-fat diets on weight loss and cardiovascular risk factors: a meta-analysis of randomized controlled trials. Arch Intern Med, 2006. 166(3): p. 285-93; Anderson J W, Brinkman-Kaplan V L, Lee H, Wood C L. Relationship of weight loss to cardiovascular risk factors in morbidly obese individuals. J Am Coll Nutr, 1994. 13(3): p. 256-61).

### SUMMARY OF THE INVENTION

The foregoing needs and other needs and objectives that will become apparent for the following description are achieved in the present invention which comprises, a composition and methods of a diet supplement for use in a mammal. The diet supplement of the present invention comprises a combination of at least, an extract of Cissus Quadrangularis stem and leaf, an extract of Green Tea leaf, and an effective amount of Caffeine Anhydrous to support weight loss, such that the amount of Caffeine Anhydrous is related to the amount of caffeine contained in the extract of Green Tea. The present invention may further comprise Chromium Polynicotinate, an extract of Soy Bean Albumin, Pyridoxine Hydrochloride, L-selenomethionine, Cyanocobalmin, Yohimbine HCl and Folic Acid.

According to an embodiment, the diet supplement is provided in a caplet form, which may be consumed several times per day. For instance, the diet supplement may comprise at least a 2 caplet serving; each serving being suitable for consumption at least 2 times daily, e.g., before daily meals. In another embodiment, the diet supplement is provided in the form of liquid capsules, comprising at least a 2 liquid capsule serving; each liquid capsule being suitable for consumption at least 2 times daily. In this manner, the diet supplement may cause weight loss and reduce the waist circumference of an individual, e.g., a human or an animal, for an extended period of time, e.g., all day.

Another aspect of the invention provides, by the consumption of the diet supplement, a method for causing weight loss, reducing the waist circumference of a mammal. For example, the present invention provides a method for promoting weight loss, comprising the step of administering to a mammal a diet supplement that comprises at least an extract of Cissus Quadrangularis stem and leaf, an extract of Green Tea and an effective amount of Caffeine Anhydrous. The method may include the step of administering the diet supplement to a human or animal at least once daily.

### DESCRIPTION OF THE INVENTION

In the following description, for the purposes of explanation, numerous specific details are set forth in order to provide a thorough understanding of the present invention. It will be apparent, however, to one skilled in the art that the present invention may be practiced without one or more of these specific details.

The present invention is directed towards a composition comprising at least an extract of Cissus Quadrangularis stem and leaf and an extract of Green Tea and an effective amount of Caffeine Anhydrous. According to various embodiments, the present invention is directed towards a diet supplement for promoting weight loss in a mammal. More specifically, the present invention is directed towards a composition containing an effective amount of caffeine wherein the amount of caffeine contained in the extract of green tea fluctuates.

Anhydrous Caffeine

Caffeine is a naturally occurring xanthine alkaloid found in some plants where it serves as a natural pesticide. In humans, however, it may have numerous beneficial effects, the most common of which uses caffeine as a supplement to the central nervous system. In this capacity, it is used as a stimulant and performance enhancer. A meta-analysis complied from forty double-blind studies supports the use of caffeine to increase physical endurance (Doherty M, Smith P M. Effects of caffeine ingestion on exercise testing: a meta-analysis. Int J Sport Nutr Exerc Metab, 2004. 14(6): p. 626-46; Graham T E, Hibbert E, Sathasivam P. Metabolic and exercise endurance effects of coffee and caffeine ingestion. J Appl Physiol, 1998. 85(3): p. 883-9; Kovacs E M, Stegen J, Brouns F. Effect of caffeinated drinks on substrate metabolism, caffeine excretion, and performance. J Appl Physiol, 1998. 85(2): p. 709-15).

Caffeine is also widely used to control weight, which may occur through multiple mechanisms. Significant weight loss related to caffeine supplementation has been observed in obese women (Yoshida T, Sakane N, Umekawa T, Kondo M. Relationship between basal metabolic rate, thermogenic response to caffeine, and body weight loss following combined low calorie and exercise treatment in obese women. Int J Obes Relat Metab Disord, 1994. 18(5): p. 345-50.) which may be, at least in part, due to increased lipolysis as fat is metabolized (Jung R T, Shetty P S, James W P, Barrand M A, Callingham B A. Caffeine: its effect on catecholamines and metabolism in lean and obese humans. Clin Sci (Lond), 1981. 60(5): p. 527-35.). Caffeine has additionally been shown to increase the basal metabolic rate (Roberts A T, de Jonge-Levitan L, Parker C C, Greenway F. The effect of an herbal supplement containing black tea and caffeine on metabolic parameters in humans. Altern Med Rev, 2005. 10(4): p. 321-5.) wherein this also adds to its weight-lowering effects via increased energy expenditure.

Biochemically, caffeine, as it is structurally similar to adenosine, binds to, but does not activate, adenosine receptors which are normally activated by adenosine to induce sleep (Shi D, Nikodijevic O, Jacobson K A, Daly J W. Chronic caffeine alters the density of adenosine, adrenergic, cholinergic, GABA, and serotonin receptors and calcium channels in mouse brain. Cell Mol Neurobiol, 1993. 13(3): p. 247-61.). Thus, caffeine acts as a stimulant. By antagonizing certain adenosine receptors, caffeine has the effect of increasing levels of intracellular cyclic AMP (cAMP), an important signaling molecule involved in many metabolic processes including thermogenesis (Ramkumar V, Bumgarner J R, Jacobson K A, Stiles G L. Multiple components of the A1 adenosine receptor-adenylate cyclase system are regulated in rat cerebral cortex by chronic caffeine ingestion. J Clin Invest. 1988, 82(1):242-7; Biaggioni I, Paul S, Puckett A, Arzubiaga C. Caffeine and theophylline as adenosine receptor antagonists in humans. J Pharmacol Exp Ther. 1991, 258(2):588-93). Caffeine also increases cAMP levels by inhibiting phosphodiesterases which degrade cAMP (Leblanc J, Richard D, Racotta I S. Metabolic and hormone-related responses to caffeine in rats. Pharmacol Res. 1995, 32(3):129-33). These actions of caffeine lead to an increase in the release of epinephrine and norepinephrine (Thong F S, Graham T E. Caffeine-induced impairment of glucose tolerance is abolished by beta-adrenergic receptor blockade in humans. J Appl Physiol. 2002, 92(6):2347-52; Smith A, Brice C, Nash J, Rich N, Nutt D J. Caffeine and central noradrenaline: effects on mood, cognitive performance, eye movements and cardiovascular function. J Psychopharmacol. 2003, 17(3):283-92). Since epinephrine and norepinephrine use cAMP for signaling, increased levels of cAMP will yield increased adrenergic signaling thereby inducing lipolysis.

In an embodiments of the present invention which is set forth in greater detail below in Example 1, the diet supplement includes an effective amount of Anhydrous Caffeine for supporting weight loss. In an embodiment, the amount of Caffeine Anhydrous is related, e.g., inversely proportional, to the amount of caffeine contained in an extract of Green Tea. Since the percentage of caffeine contained in extracts of Green Tea may vary significantly, an effective amount of Caffeine Anhydrous is qualified in relation to the specific amount of caffeine contained in an extract of Green Tea. In another aspect, a serving of the diet supplement may include from about 0.01 g to about 1 g of Anhydrous Caffeine. Three preferred dosages, in a serving of said diet supplement, comprise about 0.2000 g, 0.2500 or 0.3000 g, respectively, of Anhydrous Caffeine.

Cissus Quadrangularis Stem and Leaf Extract

Cissus Quadrangularis is a plant indigenous to India where it is part of traditional medicine. Extracts of Cissus Quadrangularis contain sterols, vitamin C, and tannins with antimicrobial in addition to antioxidant activity (Chidambara Murthy K N, Vanitha A, Mahadeva Swamy M, Ravishankar G A. Antioxidant and antimicrobial activity of Cissus quadrangularis L. J Med Food, 2003. 6(2): p. 99-105). The antioxidant activity has been proposed to be one of the mechanisms of protection against tissue injury (Jainu M, Mohan K V, Devi C S. Protective effect of Cissus quadrangularis on neutrophil mediated tissue injury induced by aspirin in rats. J Ethnopharmacol, 2006. 104(3): p. 302-5). With respect to Cissus Quadrangularis as a weight loss agent, clinical studies have shown that a group taking an extract of Cissus Quadrangularis for 6-weeks lost comparatively more weight, had lower cholesterol, LDL and fasting blood glucose levels. The experimental group also displayed increased HDL levels as compared to a placebo group (Oben J E, Mandob D, Fomekong G, Momo C. The effect of an extract of Cissus quadrangularis (Cylaris™) on weight and serum lipids in obese patients in Cameroon: a randomized double-blind clinical trial. Presented at Paris Anti-Obesity Therapies. May 2006).

In an embodiment of the present invention which is set forth in greater detail below in Example 1, the diet supplement may include an extract of Cissus Quadrangularis. A serving of the diet supplement may include from about 0.01 g to about 1 g of an extract of Cissus Quadrangularis. The preferred dosage, in a serving of said diet supplement, comprises about 0.1500 g of Cissus Quadrangularis Extract.

### Green Tea Extract (Polyphenols, Catechins and ECGC)

The active compounds of Green Tea are a family of polyphenols wherein tannins are the largest of the group. The most active specific compound is epigallocatechin gallate (ECGC) which makes up 10-50% of the total Catechins. Green tea also contains a percentage of caffeine, although the percentage of caffeine contained in extracts of green tea fluctuates significantly.

Green Tea mainly acts in a beneficial way through the polyphenol's antioxidant activities as evidenced by several laboratory studies. One clinical study has shown that ingestion of an extract of green tea results in a rapid increase in plasma antioxidant activity (Benzie I F, Szeto Y T, Strain J J, Tomlinson B. Consumption of green tea causes rapid increase in plasma antioxidant power in humans. Nutr Cancer, 1999. 34(1): p. 83-7.). Moreover, Green Tea has also been shown to be effective in aiding weight loss (Chantre P, Lairon D. Recent findings of green tea extract AR25 (Exolise) and its activity for the treatment of obesity. Phytomedicine, 2002. 9(1): p. 3-8.). This effect may be due to two activities. Green Tea both reduces fat digestion and as a second pathway for weight loss linked to Green Tea, it increases energy expenditure (Berube-Parent S, Pelletier C, Dore J, Tremblay A. Effects of encapsulated green tea and Guarana extracts containing a mixture of epigallocatechin-3-gallate and caffeine on 24 h energy expenditure and fat oxidation in men. Br J Nutr, 2005. 94(3): p. 432-6.). Fat stores may provide the energy necessary for the increase in energy expenditure via the oxidation of fat (thermogenesis) (Choo J J. Green tea reduces body fat accretion caused by high-fat diet in rats through beta-adrenoceptor activation of thermogenesis in brown adipose tissue. J Nutr Biochem, 2003. 14(11): p. 671-6; Dulloo A G, Duret C, Rohrer D, Girardier L, Mensi N, Fathi M, Chantre P, Vandermander J. Efficacy of a green tea extract rich in catechin polyphenols and caffeine in increasing 24-h energy expenditure and fat oxidation in humans. Am J Clin Nutr, 1999. 70(6): p. 1040-5.). The thermogenic activity of Green Tea may additionally be greatly enhanced by synergistic cooperation with caffeine (Dulloo A G, Seydoux J, Girardier L, Chantre P, Vandermander J. Green tea and thermogenesis: interactions between catechin-polyphenols, caffeine and sympathetic activity. Int J Obes Relat Metab Disord, 2000. 24(2): p. 252-8.).

The mechanism of action of Green Tea may be, at least partially, due to an increase in norepinephrine. Catechins are known to inhibit catechol-O-methyl-transferase (COMT), an enzyme that degrades norepinephrine (Borchardt R T, Huber J A. Catechol O-methyltransferase. 5. Structure-activity relationships for inhibition by flavonoids. J Med Chem, 1975. 18(1): p. 120-2.). In turn, norepinephrine inhibits degradation of cAMP. Increasing norepinephrine levels by inhibition of norepinephrine uptake results in increased weight loss in both lean and obese mice (Billes S K, Cowley M A. Inhibition of Dopamine and Norepinephrine Reuptake Produces Additive Effects on Energy Balance in Lean and Obese Mice. Neuropsychopharmacology. 2006 Jul. 12). Increased norepinephrine levels result in increased interaction with adrenergic receptors which are known to regulate lipolysis (Lafontan M, Barbe P, Galitzky J, Tavernier G, Langin D, Carpene C, Bousquet-Melou A, Berlan M. Adrenergic regulation of adipocyte metabolism. Hum Reprod. 1997, 12 Suppl 1:6-20).

In an embodiment of the present invention which is set forth in greater detail below in Example 1, the diet supplement includes an extract of Green Tea Dry Leaf (camellia sinensis). A serving of the diet supplement may include from about 0.01 g to about 1 g of an extract of Green Tea Dry Leaf (camellia sinensis). The preferred dosage, in a serving of said diet supplement, comprises about 0.1222 g to about 0.46000 g of an extract of Green Tea Dry Leaf (camellia sinensis).

Chromium Polynicotinate

Chromium is an essential trace mineral that is used to control blood sugar levels by aiding insulin binding, wherein it can aid in the control weight of reduction. Chromium, however, is poorly absorbed by the body and must therefore be combined with a more efficiently absorbed compound such as niacin (found in Polynicotinate). Chromium likely exerts its main function as a component or co-factor of the glucose tolerance factor, which is involved in insulin sensitivity.

Chromium has been shown clinically to increase lean mass (Bahadori B, Wallner S, Schneider H, Wascher T C, Toplak H. Effect of chromium yeast and chromium picolinate on body composition of obese, non-diabetic patients during and after a formula diet. Acta Med Austriaca. 1997; 24(5):185-7) and reduce body fat when combined with exercise (Grant K E, Chandler R M, Castle A L, Ivy J L. Chromium and exercise training: effect on obese women. Med Sci Sports Exerc. 1997 August; 29(8):992-8.). Chromium has also been shown to increase HDL, (‘good’) cholesterol levels (Riales R, Albrink M J. Effect of chromium chloride supplementation on glucose tolerance and serum lipids including high-density lipoprotein of adult men. Am J Clin Nutr 1981; 34:2670-8.). Both of these actions may lead to a reduction in weight loss and a change in the LDL/HDL ratio.

In an embodiment of the present invention which is set forth in greater detail below in Example 1, the diet supplement may include Chromium Polynicotinate. A serving of the diet supplement may include from about 0.01 mg to about 10 mg of Chromium Polynicotinate. The preferred dosage, in a serving of said diet supplement, comprises about 1.5 mg of Chromium Polynicotinate.

Yohimbine

Yohimbine is a naturally occurring alkaloid derived from the African tree, Pausinysatlia yohimbe. Traditionally, Yohimbine has been used as an aphrodisiac in traditional medicine and additionally as a treatment for erectile dysfunction (MacKay D. Nutrients and botanicals for erectile dysfunction: examining the evidence. Altern Med Rev. 2004 March; 9(1):4-16.). Furthermore, Yohimbine has been shown through research to increase the amount of non-esterified fatty acids (NEFAs) in the bloodstream, as a product of lipolysis in both lean and obese individuals (Berlan M, Galitzky J, Riviere D, Foureau M, Tran M A, Flores R, Louvet J P, Houin G, Lafontan M. Plasma catecholamine levels and lipid mobilization induced by Yohimbine in obese and non-obese women. Int J. Obes. 1991 May; 15(5):305-15; Galitzky J, Taouis M, Berlan M, Riviere D, Garrigues M Lafontan M. Alpha 2-antagonist compounds and lipid mobilization: evidence for a lipid mobilizing effect of oral Yohimbine in healthy male volunteers. Eur J Clin Invest. 1988 December; 18(6):587-94.). Additionally this effect persists for upwards of 14 days, indicating that a rapid tolerance to Yohimbine does not develop (Galitzky J, Taouis M, Berlan M, Riviere D, Garrigues M Lafontan M. Alpha 2-antagonist compounds and lipid mobilization: evidence for a lipid mobilizing effect of oral Yohimbine in healthy male volunteers. Eur J Clin Invest. 1988 December; 18(6):587-94.). In addition to its effects on lipolysis, Yohimbine is also an appetitie suppressant, and has been shown to decrease energy intake in both lean and obese mice (Currie P J, Wilson L M. Yohimbine attenuates clonidine-induced feeding and macronutrient selection in genetically obese (ob/ob) mice. Pharmacol Biochem Behav. 1992 December; 43(4):1039-46.) Researchers also found that in a 3-week, 20-obese-female subject study, wherein the subjects were restricted to a 1000 calories per day diet, that 20 mg of Yohimbine administered daily increased weight loss by an additional 3 pounds over the placebo group (Kucio C, Jonderko K, Piskorska D. Does Yohimbine act as a slimming drug? Isr J Med Sci. 1991 October; 27(10):550-6.).

In an embodiment of the present invention which is set forth in greater detail below in Example 1, the diet supplement may include Yohimbine HCl. A serving of the diet supplement may include from about 0.01 mg to about 10 mg of Yohimbine HCl. The preferred dosage, in a serving of said diet supplement, comprises about 4.5 mg of Yohimbine HCl.

In an embodiment, the present invention may comprise a diet supplement containing an extract of Cissus Quadrangularis, an extract of Green Tea Dry Leaf, an effective amount of Caffeine Anhydrous, and Chromium Polynicotinate. The present embodiment may further comprise at least one of an Extract of Soy Bean Albumin, Pyridoxine Hydrochloride, L-selenomethionine, Cyanocobalmin, Yohimbine HCl and Folic Acid wherein said diet supplement may be administered to mammal—at least 2 times per day.

The diet supplement according to the present invention provides a method for causing weight loss and reducing the waist circumference of a mammal. Advantageously, consumption of the diet supplement is combined with a reduced calorie diet and a program of regular exercise.

According to various embodiments of the present invention, the diet supplement may be consumed in any form. For instance, the dosage form of the diet supplement may be provided as, e.g., a powder beverage mix, a liquid beverage, a ready-to-eat bar or drink product, a capsule, a liquid capsule, a tablet, a caplet, or as a dietary gel. The preferred dosage forms of the present invention are as a caplet or as a liquid capsule.

Preferably, the diet supplement is consumed by an individual in accordance with the following method: as a diet supplement, a serving of said diet supplement may be taken in conjunction with 8 fluid oz. of a liquid medium two times daily wherein each serving is comprised of 2 caplets or liquid capsules. Said serving may be consumed approximately 30 to 60 minutes before each meal, preferably in the morning and afternoon. In this manner, the diet supplement may cause weight loss, reduce the waist circumference, ameliorate blood cholesterol levels, improve blood triglyceride levels, and improve the fasting blood glucose profile of a mammal for an extended period of time, e.g., all day.

Furthermore, the dosage form of the diet supplement may be provided in accordance with customary processing techniques for herbal and dietary supplements in any of the forms mentioned above. Additionally, the diet supplement set forth in the example embodiment herein may contain any appropriate number and type of excipients, as is well known in the art.

The present diet supplement or those similarly envisioned by one of skill in the art, may be utilized in compositions and methods to cause weight loss, ameliorate blood cholesterol levels, improve blood triglyceride levels, improve the fasting blood glucose profile and reduce the waist circumference of and individual, e.g., a human or an animal, in a formulation designed to be consumed at least two times per day.

Although the following example illustrates the practice of the present invention in one of its embodiments, the example should not be construed as limiting the scope of the invention. Other embodiments will be apparent to one of skill in the art from consideration of the specifications and example.

### Example 1

A diet supplement for causing weight loss and reducing the waist circumference of an individual is provided, the diet supplement comprising from about 0.2000 g to about 0.3000 g of Caffeine Anhydrous, about 0.1500 g of an extract of Cissus Quadrangularis, about 0.1222 g to about 0.46000 g of an extract of Green Tea Dry Leaf, about 0.0500 g of an extract of Soy Bean Albumin, about 0.0500 g of Pyridoxine Hydrochloride, about 0.0120 g of L-selenomethionine, about 0.0050 g of Cyanocobalmin, about 0.0015 g of Chromium Polynicotinate, 0.0045 g of yohimbine HCl and about 0.0004 g of Folic Acid.

Directions: As a diet supplement, 2 caplets are administered with an 8 oz. glass of water two (2) times daily. Each two caplet or liquid capsule serving may be consumed approximately 30 to 60 minutes before meals, preferably in the morning and afternoon.

### EXTENSIONS AND ALTERNATIVES

In the foregoing specification, the invention has been described with specific an embodiment thereof, however, it will be evident that various modifications and changes may be made thereto without departing from the broader spirit and scope of the invention.

What is claimed:

1. A diet supplement for supporting weight loss comprising at least, an extract of Cissus quadrangularis stem and leaf, an extract of and green tea leaf, an effective amount of caffeine anhydrous and yohimbine HCl.

2. The diet supplement of claim 1, wherein the effective amount of caffeine anhydrous is inversely proportional to an amount of caffeine contained within the extract of green tea leaf.

3. The diet supplement of claim 2, wherein the effective amount of caffeine anhydrous is a maximum of 0.3000 g.

4. The diet supplement of claim 1, wherein the total amount of caffeine is a maximum of 0.3500 g.

5. The diet supplement of claim 1, further comprising at least one of chromium polynicotinate, an extract of soy bean albumin, pyridoxine hydrochloride, L-selenomethionine, cyanocobalmin and folic acid.

6. The diet supplement of claim 5, further comprising about chromium polynicotinate.

7. A method for promoting weight loss comprising the step of providing to a mammal a diet comprising the diet supplement of claim 1.

8. The method of claim 7, wherein the diet supplement further comprises at least one of chromium polynicotinate, an extract of soy bean albumin, pyridoxine hydrochloride, L-selenomethionine, cyanocobalmin and folic acid.

9. The method of claim 7 wherein the mammal is a human.

10. The diet supplement of claim 6, wherein the total amount of chromium polynicotinate is about 0.0150 g.

11. A diet supplement for causing weight loss comprising from about 0.2000 g to about 0.3000 g of caffeine anhydrous, about 0.1500 g of an extract of Cissus quadrangularis, about 0.1222 g to about 0.46000 g of an extract of green tea dry leaf, about 0.0500 g of an extract of soy bean albumin, about 0.0500 g of pyridoxine hydrochloride, about 0.0120 g of L-selenomethionine, about 0.0050 g of cyanocobalmin, about 0.0015 g of chromium polynicotinate, 0.0045 g of yohimbine HCl and about 0.0004 g of folic acid.

12. A method for promoting weight loss comprising the step of providing to a mammal a diet comprising the diet supplement of claim 11.

13. The method of claim 13 wherein the mammal is a human.
